(19) United States
(12) Patent Application Publication
(10) Pub. No.: US 2022/0257395 A1
(43) Pub. Date: Aug. 18, 2022

(54) STENT

(71) Applicant: John I. Shipp, Jacksonville, FL (US)

(72) Inventor: John I. Shipp, Jacksonville, FL (US)

(21) Appl. No.: 17/672,281

(22) Filed: Feb. 15, 2022

Related U.S. Application Data
(60) Provisional application No. 63/149,405, filed on Feb. 15, 2021.

(51) Int. Cl. A61F 2/93; A61F 2/94; A61F 2/958; A61F 2/844; A61L 31/06

(52) U.S. Cl. CPC A61F 2/93; A61F 2/94; A61F 2210/0004; A61F 2/844; A61L 31/06; A61F 2/958

(57) ABSTRACT

A fully absorbable stent for treating lesions in vessels in the body has a flexible covering and expandable belts. The device is formed from very low inflammation response materials that are fully absorbed in 3 to 12 months. The expandable belts provide radial forces for keeping the vessel open following balloon angioplasty.

This application claims priority to U.S. Patent Application Ser. No. 63/149,405 filed on Feb. 15, 2021, the contents of which are hereby incorporated by reference in their entirety.

### BACKGROUND OF THE INVENTION

### Field of the Invention

The present invention relates generally to an absorbable stent, in particular, to a stent with an outer covering and expandable belts within the covering. An inflatable balloon can be used to locate and expand the expandable belts at a lesion location to re-open the blood vessel.

### Technical Background

Vascular artery disease causes blood vessels to become narrow owing to intravascular lesions or plaque comprising lipids and calcified materials. In the case of coronary artery disease (CAD), because of these lesions the oxygen supply is reduced to the heart muscle and can result in a heart attack, a myocardial infarction (MI), which can result in death. The primary treatment of CAD initially was implantation of coronary artery bypass grafts (CABG), performed by cardiac surgeons. Mortality and morbidity are rather high mainly due to the extreme invasiveness of the procedure.

In the 1970s, a less invasive treatment was developed using balloons. These devices were inserted through a small incision in the femoral artery. Balloon angioplasty is used to widen an artery that has become narrowed with lesions. The balloon is inserted in the artery, guided to the lesion site, and inflated to open the artery by compressing the plaque. The procedure is called Percutaneous Transluminal Coronary Angioplasty (PTCA). In 3 to 6 months following balloon angioplasty, 40 to 50% of coronaries arteries are affected by re-narrowing (restenosis) of the blood vessel after it has been opened by balloon angioplasty.

In 1986, to counter restenosis, metal stents were first used to supply a permanent radial force on the artery wall attempting to maintain vessel patency. First a guide wire is inserted through the lesion followed by a shaping balloon that is expanded to make room for the metal stent which is either self-expanded or expanded using a second balloon. Both balloon and stent devices employ radio-opaque markers to insure accurate device placement under fluoroscopy. While delaying restenosis as compared to balloon angioplasty alone, unfortunately, the bare metal stent (BMS) elicits significant foreign body response resulting in inflammation, prolapse, scarring, and thrombosis. Note that the use of the word restenosis can be misinterpreted and leave the impression that it is a reoccurrence of the mechanisms that causes the original stenosis. The two biological processes are mostly unrelated. The original lesions form over decades driven by diet, smoking, and genetics. “Restenosis” is caused by the body's response to artery wall injury by the inflation balloons and stents, and foreign body response to the implant.

To counter the foreign body response and in particular to deter scar tissue from growing in the artery lumen (negative remolding), drug eluting stents (DES) were developed in late 1990s. Immune suppression and antiproliferative drugs are attached to the metal stent struts using a bio-absorbable coating. The drugs are released as the coating is absorbed, usually over a period of 90 to 120 days. After the coating is absorbed and the drug is eluted, clots can form around the stent and cause adverse events including MI. Regimens of blood thinner and antiplatelet drugs are often prescribed for up to a year post operatively following implant of a DES. In addition to the expense of the regimen, these anticoagulant drugs significantly increase the likelihood of hematomas and strokes. These DES decreased the restenosis in the first year from 40% to 10% but late term thrombosis remains problematic.

More recently drug eluting balloons accompanied by a BMS have been developed. The drug bonded to the balloon is quickly released (in a few seconds) with its carrier onto the vessel wall followed by insertion of a BMS. The drug is released from the carrier over a 2-3-month period. The drug coated balloon/BMS combination has found some success. In a sense the drug coated balloon (DCB) and the DCS are two pathways to the same end, time release of drugs to mediate inflammation caused by damage to the artery wall and foreign body response to the BMS.

Attempting to eliminate the need for complicated drug release, balloon/stent and DES designs, researchers in Japan developed a fully bio-absorbable vascular stent formed from Poly L Latic Acid, PLLA. The polymer proved to be inflammatory, prompting Abbott Vascular to develop a drug eluting, fully absorbable stent (BVS) also formed from PLLA. The drug used in the Abbot BVS device is an immune suppressor overcoated with Poly D, L Lactide. The device did not perform well compared with metallic drug eluting stents. Both bare metal stents and bare PLLA stents have been shown to be highly inflammatory so it is not surprising that inflammation would return after the drug coating is absorbed and the drug eluted, leaving again bare stents.

Current designs of both metal and polymer stents comprise a series of struts and apertures, usually with 80-90% porosity, that allow the devices to expand from their initial insertion profile (typically 6 F) to the diameter of the targeted vessel. In the case of main coronary arteries such as left anterior descending (LAD), the diameter is typically 2-4 mm. Thus, the stent must be expandable up to 2.5×, which foreshortens the stent owing to the interconnected mesh apertures. These strut-aperture designs allow some lesions, depending on the degree of calcification, to re-migrate toward the interior of the vessel, a process known as prolapse.

Peripheral artery disease (PAD) is often characterized by long lesions (4-10 cm) comprising heavily calcified material that renders balloon and stenting almost impossible. These lesions are often debulked by rotating mechanical cutters or laser ablation prior to the use of balloon angioplasty. Stents are sometimes inserted in the target area attempting to prolong the vessel patency. Vessel wall injury during the debulking process and inflammation cause by foreign body reaction to the metallic stent material causes the vessel to remodel negatively, (inward) leading to restenosis. The current invention comprises a stent with very low inflammatory response that allows the vessel to heal naturally, with or without debulking, thus allowing positive or outward remodeling, maximizing patency. The stent materials of the current invention have been shown to be pulled out of blood circulation within 30 days covered by full thickness intima and producing positive vessel remodeling. The stent materials are then hydrolyzed into water and carbon dioxide outside the blood flow, in about 120 days.

PAD and CAD are gradual conditions that can take decades to become symptomatic. The chemistry of the blood-vascular interface is complicated. The complexity and slowness of the process make scientific study difficult, hence the depth of knowledge is lacking. Current treatment modes, both balloon and stent angioplasty with and without eluting drugs trigger adverse artery reactions causing inflammation which leads to restenosis and thrombus development.

What is needed then is a stent constructed from a bio-absorbable polymer that causes minimal inflammation without the need for drug elution or other additives and allows for positive intima remodeling before being fully absorbed, preferably in 3 to 12 months. The stent must supply adequate radial force for patency, be adjustable to assure that the vessel is opened to adequate patency for all types of plaque, not allow prolapse of lesion material, not encourage inward or negative remodeling, and not have so much radial force to significantly damage the artery. The stent should, preferably, be expandable from 1.7-2 mm (5-6 F) to 4-8 mm diameter without changing the length. The stent should be self, mechanical, or balloon expandable.

### SUMMARY OF THE INVENTION

The present invention is directed to an absorbable stent to be inserted into a lumen of a blood vessel that includes a flexible covering having a central opening extending between a first end and a second end, and at least one expanding belt disposed within the central opening between the first end and the second end, the expanding belt having an inside surface and an outside surface and expanding radially in response to pressure exerted on the inside surface of the belt.

In some embodiments, the at least one expanding belt comprises three expanding belts, the expanding belts disposed evenly within the central opening of the flexible covering.

In some embodiments, the at least one expanding belt has a plurality of teeth disposed along the outside surface to engage a pawl disposed on the at least one expanding belt.

In other embodiments, the teeth have a first surface with an angle of α1 to the outside surface and a second surface having and angle of α2 to the outside surface.

In yet another aspect, the invention is directed to a method of inserting an absorbable stent in a blood vessel that includes providing the stent and a catheter having an expandable balloon, the stent having a flexible covering with a central opening extending between a first end and a second end and at least one expanding belt disposed within the central opening between the first end and the second end, the expanding belt having an inside surface and an outside surface and expanding radially in response to pressure exerted on the inside surface of the belt, locating the stent on the expandable balloon on the catheter, locating the stent and the expandable balloon at a lesion site in a blood vessel through a vessel opening, expanding the expandable balloon, thereby expanding the stent at the lesion site, deflating the expandable balloon, and removing the expandable balloon from the blood vessel.

In yet another aspect, the invention is directed to an absorbable stent to be inserted into a lumen of a blood vessel that includes a flexible covering having a central opening extending between a first end and a second end; and a plurality of expanding belts disposed within and spaced along the central opening between the first end and the second end, each of the plurality of expanding belts having an inside surface and an outside surface and having a plurality of teeth disposed along the outside surface to engage a pawl disposed on each of the plurality of expanding belts and each of the plurality of expanding belts expanding radially in response to pressure exerted on the inside surface of the belt.

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

It is to be understood that both the foregoing general description and the following detailed description of the present embodiments of the invention, and are intended to provide an overview or framework for understanding the nature and character of the invention as it is claimed. The accompanying drawings are included to provide a further understanding of the invention, and are incorporated into and constitute a part of this specification. The drawings illustrate various embodiments of the invention and together with the description serve to explain the principles and operations of the invention.

### BRIEF DESCRIPTION OF THE DRAWINGS

FIG. 1 is a perspective view of one embodiment of a stent according to the present invention;

FIG. 2 is a perspective view of an expandable belt of the stent of FIG. 1;

FIG. 3 is a cross section view of the buckle and pawl of the expandable belt of the stent of FIG. 1;

FIG. 4 is an end view of the stent fully deployed in a blood vessel;

FIG. 5 is an elevational side view of the expandable belt used in both embodiments of the stent in an initial configuration;

FIG. 6 is an elevational side view of the expandable belt used in both embodiments of the stent in a fully deployed configuration;

FIG. 7 is a partial cross section view of the stent in FIG. 1 disposed within the blood vessel at a lesion and prior to the expandable belts being expanded by the balloon catheter;

FIG. 8 is a partial cross section view of the stent in FIG. 1 disposed within the blood vessel at the lesion and after the expandable belts have been expanded by the balloon catheter;

FIG. 9 is a perspective view of a second embodiment of a stent according to the present invention;

FIG. 10 is a perspective view another embodiment of a stent according to the present invention; and

FIG. 11 is a perspective view of the stent in FIG. 10 disposed with the aperture in the stent at a branch site.

### DETAILED DESCRIPTION OF THE PREFERRED EMBODIMENTS

Reference will now be made in detail to the present preferred embodiment(s) of the invention, examples of which are illustrated in the accompanying drawings. Whenever possible, the same reference numerals will be used throughout the drawings to refer to the same or like parts.

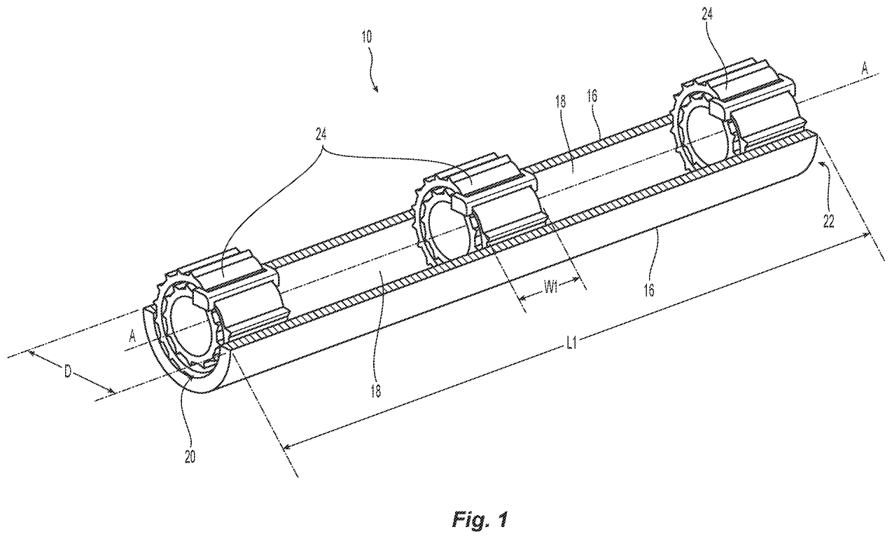

Referring to FIGS. 1-8 , there is an absorbable stent 10 that is to be inserted into a lumen 12 of a blood vessel 14. The stent 10 has a flexible covering 16 and a central opening 18 that extends between a first end 20 and a second end 22. The stent 10 also includes a plurality of expandable belts 24 that are disposed within the central opening 18 of the flexible covering 16. The plurality of expandable belts 24 are spaced along a length L of the flexible covering 16. Typically, the length L1 of a stent is between 10 and 30 mm. The expandable belts 24 preferably have a width W1 of about 1 mm in width, but could be wider or narrower for any given situation. Thus, for a stent of 10 mm and an expandable belt width of 1 mm, the distance between the expandable belts 24 is about 3.5 mm, if the expandable belts 24 are to be evenly disposed within the central opening 18. However, the spacing between the expandable belts 24 may be uneven, or the expandable belts 24 may even be disposed adjacent another. Additionally, while three expandable belts 24 are illustrated in the figures, there may be more or fewer expandable belts 24 within the central opening 18. The number of expandable belts 24 will depend on a number of factors, including the length of the stent, the length of any lesion, and the severity of the lesion, among others. It may be preferable to have one of the expandable belts 24 next to each of the first end 20 and the second end 22, as illustrated, or have some distance between the expandable belts 24 and the flexible covering 16.

The flexible covering 16 is preferably flexible in a radial direction outward from a longitudinal axis A through the central opening 18. The flexible covering 12 is preferably formed as an extruded cylinder from a flexible elastic co polymer such as Poly (l-lactide co ε caprolactone), PLC, in the molar ratio from 70:30 with IV between 1.4-2.0 dl/g after forming. However, it could be formed in other ways as well. The flexible covering 16 must be able to sustain the stent expansion without material failure. Other absorbable polymers in different mole ratios and IVs fall within the present invention.

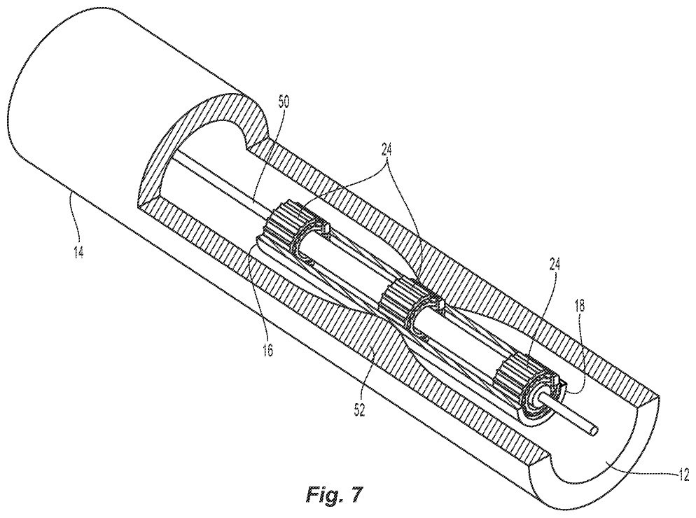

The overall diameter D of absorbable stent 10 in the collapsed or initial state (see FIG. 7) is less than 1.7 mm to accommodate a 5 French catheter system and less than 2 mm to accommodate a 6 French system, both of which are currently in common use. In the expanded state the absorbable stent 10 is in the 2-4 mm range for use in the coronary artery systems. Again, the absorbable stent 10 may be used in different areas of the body and can vary in both the collapsed and expanded diameter D.

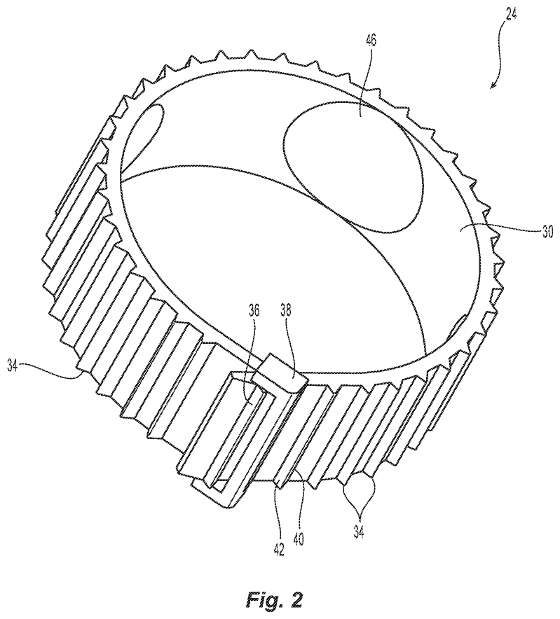

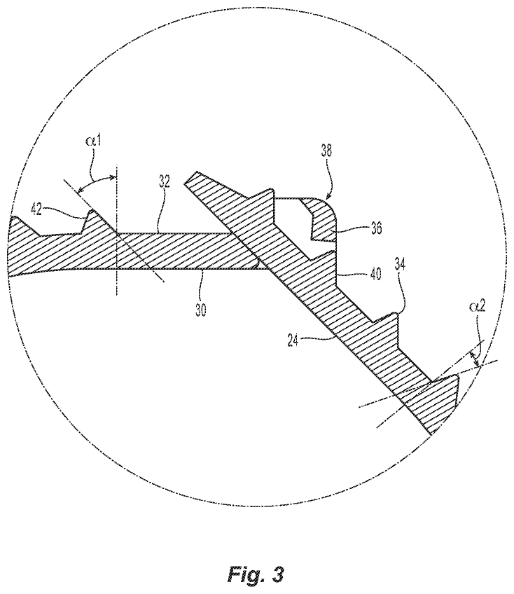

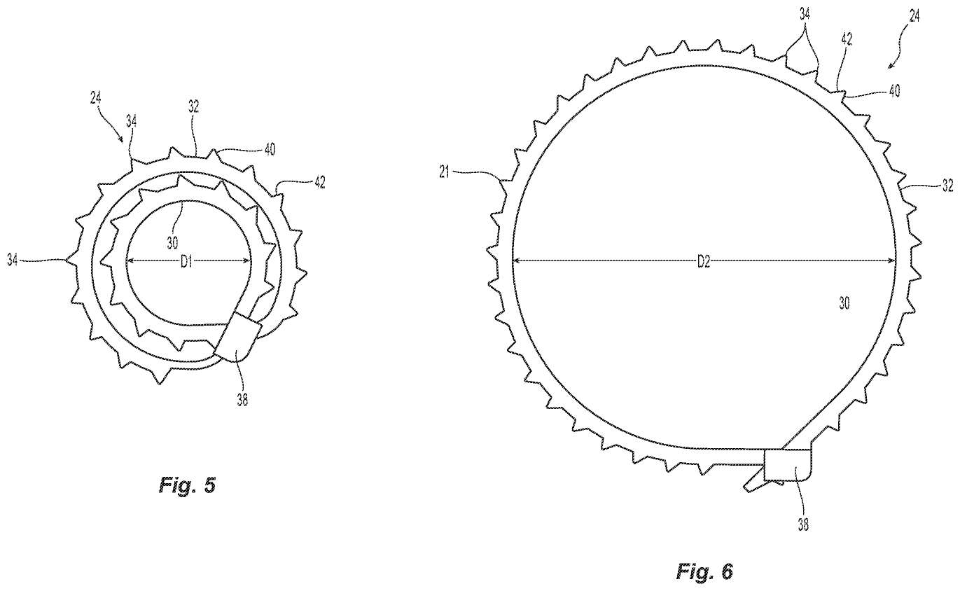

The expandable belts 24 have an inside surface 30 and an outside surface 32. The inside surface 30 of the expandable belts 24 is preferably smooth, while the outside surface 32 has plurality of teeth 34 disposed along the outside surface 32 to engage a pawl 36 within a buckle 38. See FIGS. 2 and 3. Each of the plurality of teeth 34 has a first side 40 and a second side 42. The first side 40 makes a snap angle α1 with an orthogonal axis through the expandable belt, and second side 42 makes a snap angle α2 with the orthogonal axis. For a given set of polymer mechanical properties, the plurality of teeth 34 and pawl 36, the geometries associated with the snap angle α1 dictates the force required to open the expandable belt 24 to a larger diameter. The snap angle α1 is preferably between 30 and 60 degrees. Similarly, the snap angle α2 dictates the force required to reclose the expandable belt 24 and is preferably between 50 and 85 degrees. For a given set of polymer mechanical properties, and the geometries of the teeth and pawl, the two angles are chosen to provide adequate user feedback (α1) and maximum artery wall radial force as discussed above (α2). The angle values can be calculated according to formulas well known in the art. As the expandable belt 24 is expanded, the plurality of teeth 34 pass through the buckle 38 and the pawl 36. This causes the diameter D1 of the expandable belt 24 to increase to a second diameter D2. While FIGS. 5 and 6 illustrate that the expandable belt 24 is at a maximum, the expandable belt 24 may have a smaller D2 that that illustrated in FIG. 6. The diameter D1 may also be larger or smaller than that illustrated in FIG. 5. As the expandable belt 24 expands, the plurality of teeth 34 pass through the buckle 38 and past the pawl 38. The expandable belt 24 is prevented from shrinking because the pawl 36 will engage the second side 42 of the expandable belt 24. As noted above, there is a force that would need to be overcome for the expandable belt 24 to have a decrease in the diameter. The expandable belts 24 may also have a radiopaque marker 46 that assists in identifying the location of the stent 10 under fluoroscopy or other radiography.

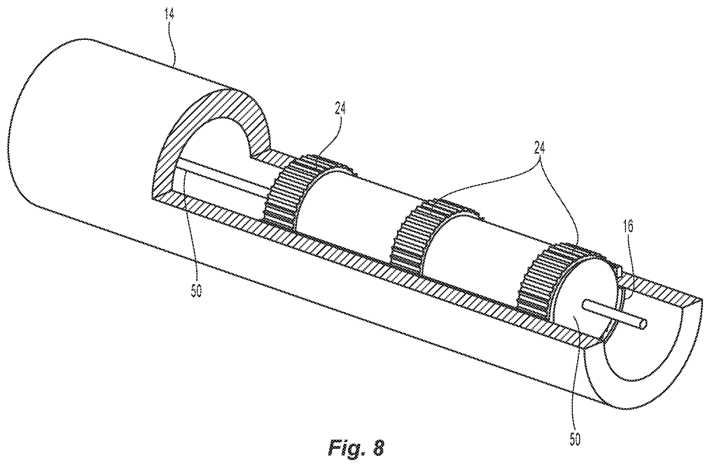

Turning to FIGS. 7 and 8, the process of expanding the expandable belt 24 (and the flexible covering 16 will be described. The stent 10 is disposed around a balloon catheter 50 and the combination of the catheter/stent. The flexible covering 16 is preferably extruded into a flexible thin wall cylindrical tube with inside diameter slight smaller than the deflated inflation balloon. This also allows for the flexible covering 16 to be stretched over the assembly, including the expandable belts 24. The assembly is then located near a lesion 52 in the lumen 12 of the blood vessel 14 using well-known techniques. When located appropriately, the balloon catheter 50 is inflated while the stent 10 remains on the balloon catheter 50. The radial forces from the inflating balloon will cause the expandable belts 24 to expand. See FIG. 8. Once the required expansion has taken place, the balloon can be deflated and removed from the patient. Closure of the access point will then be in order. The engagement of the plurality of teeth 34 by the pawl 36 will keep the blood vessel 14 open and prevent prolapse.

In one embodiment, the flexible covering 16 is formed from Poly (l-lactide co ε caprolactone), PLC, in the molar ratio from 70:30 to 95:5, lactide:caprolactone. More preferably the ratio is 70:30. The forming of the flexible covering 16 can be any one of several methods known in the art, extrusion, injection molding, or 3-D printing, for example.

With regard to the expandable belts 24, they are formed from Poly (lactide co glycolide), PLGA in the molar ratio in the range from 50:50 to 95:5, lactide:glycolide, and most preferably 82:18. Forming of the belts can be any one of several methods know in the art, injection molding or 3 D printing, for example. The materials for both the flexible covering 16 and the expandable belts 24 are non- thrombogenic, absorbable materials with minimal inflammatory response. The absorption life of the belts and covering is, preferably, 3-12 months.

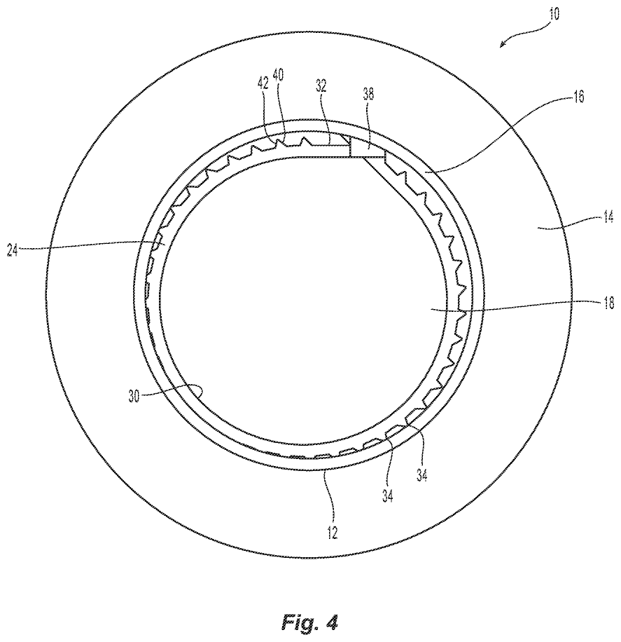

It should be noted that other procedures may take place before, during and after the use of the stent 10. For example, the target lesion 52 is located by an imaging means such as fluoroscopy. A lesion shaping balloon is inserted over a guide wire through an access point, the femoral, radial, or other artery, using a balloon catheter. The guide wire, the shaping balloon and balloon catheter are well known in the art. The balloon is inflated with a fluid, usually saline, to a pressure of between 5 and 15 atmospheres, depending on the length of the balloon and size of the lesion, restoring patency. The balloon is deflated. The artery wall often recoils to somewhat of a smaller diameter, however. The balloon catheter is exchanged, by standard sheath exchange techniques, to a stent guide catheter. The business end (distal end) of the standard guide catheter comprises a stent expansion balloon with the present invention, expandable belts 24 and the flexible covering 16 of the stent 10 surrounding the balloon in an initial or collapsed state. When the stent 10 is in place at the lesion site, the balloon is slowly inflated to ratchet up the diameter of the belts and thus expanding the elastic diameter of the covering. The teeth 34 spacing of the expandable belt 24 is chosen such that each tooth ratchet corresponds to a convenient stent diameter increase, 0.1 mm, for example. The balloon is inflated to a predetermined pressure that is chosen to be slightly above the pressure required to supply the optimum radial force and the balloon is then deflated. See FIG. 4. The stent ratchet will ratchet the diameter downward until equilibrium is reached between the wall plus the covering reaction force and the preset closing force of the belts. The catheter and balloon are removed from the body and the artery entry wound is closed by standard methods, manual compression or using a vascular closure device. During the absorption time the belts and covering are hydrolyzed into carbon dioxide and water, leaving the treated site without inflammation, scaring or thrombus.

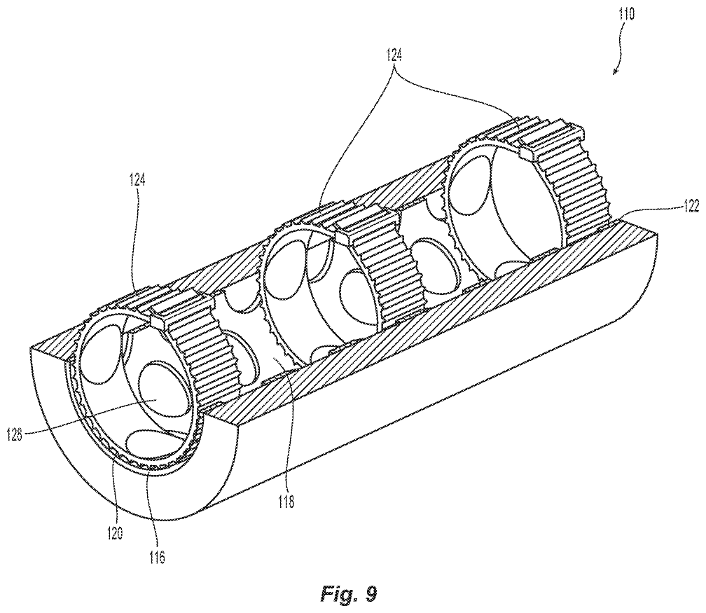

A second embodiment of a stent 100 is illustrated in FIG. 9. The stent 120 has the same main components of the first embodiment, stent 10 . That is, the stent 110 has a flexible covering 116 and a central opening 118 that extends between a first end 120 and a second end 122. The stent 110 also includes a plurality of expandable belts 124 that are disposed within the central opening 118 of the flexible covering 116. The plurality of expandable belts 124 are spaced along the flexible covering 116. In this embodiment, the flexible cover 116 is perforated with apertures 128 so as to promote endothelization of the blood vessel wall to encapsulate stent 100. The apertures 128 are shown as circular but other shapes can be employed in the invention, including but not limited to squares, triangles, stars or other such openings that provide the preferred porosity of from 0.2 to 0.9.

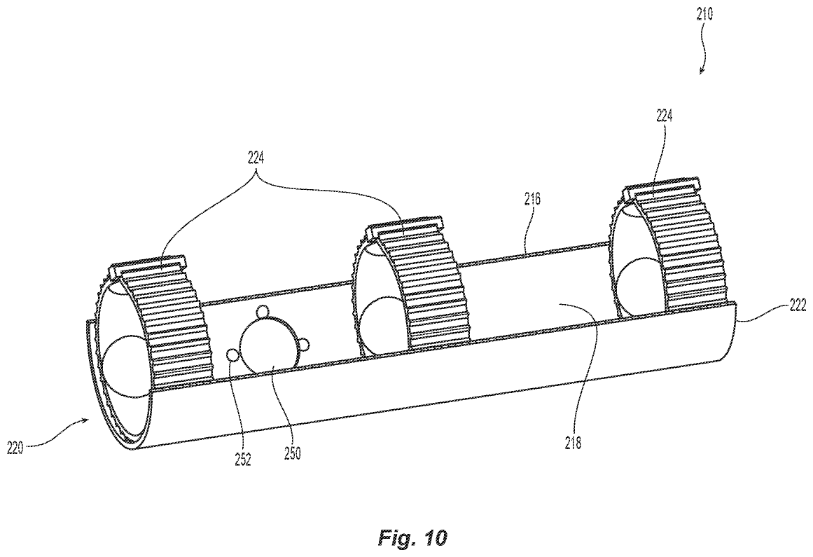

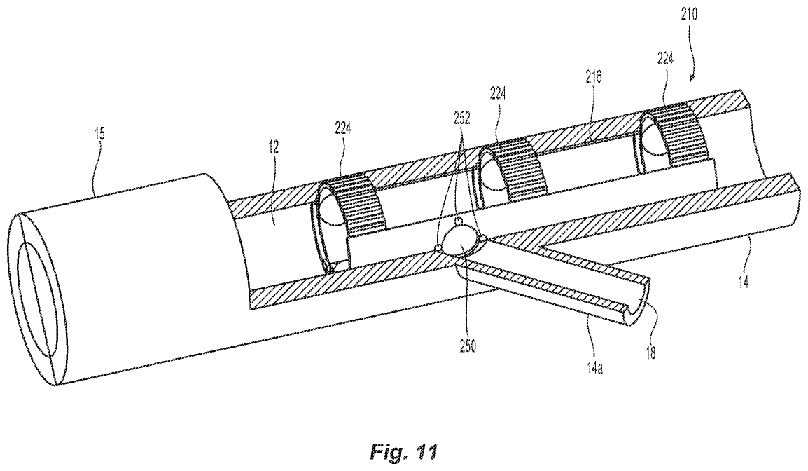

A third embodiment of a stent 210 according to the present invention is illustrated in FIGS. 10 and 11. The stent 210 has the same main components of the first embodiment, stent 10. That is, the stent 210 has a flexible covering 216 and a central opening 218 that extends between a first end 220 and a second end 222. The stent 210 also includes a plurality of expandable belts 224 that are disposed within the central opening 218 of the flexible covering 216. The plurality of expandable belts 224 are spaced along the flexible covering 216. The expandable belts 224 are the same as expandable belts 24. In this embodiment, the flexible covering 216 has an aperture 250 that allows for blood to flow from the central opening 218 to a side branch 14a of the blood vessel 14. There may also be radiopaque markers 252 to allow for locating the aperture 250 under fluoroscopy or other radiographic imaging techniques. Previous stents often block some, or all, of the side branch flow, leaving the patient with chronic chest pains.

See also AbsorbaSeal 5.6.7F vascular closure device: A good laboratory practice chronic study evaluating safety and efficacy in a healthy porcine model Sullivan A Ayuso1, R Caroline Shipp2, Bola G Aladegbami1, Delton Farquharson3, Denny Lawson4 and Randy Bassett, Vascular 2021, Vol. 0(0) 1-9.

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

I claim:

1. An absorbable stent to be inserted into a lumen of a blood vessel comprising:
a flexible covering having a central opening extending between a first end and a second end; and
at least one expanding belt disposed within the central opening between the first end and the second end, the expanding belt having an inside surface and an outside surface and expanding radially in response to pressure exerted on the inside surface of the belt.

2. The absorbable stent according to claim 1, wherein the at least one expanding belt comprises three expanding belts, the expanding belts disposed evenly within the central opening of the flexible covering.

3. The absorbable stent according to claim 1, wherein the flexible covering and the at least one expanding belt is made of absorbable materials.

4. The absorbable stent according to claim 1, wherein the at least one expanding belt has a plurality of teeth disposed along the outside surface to engage a pawl disposed on the at least one expanding belt.

5. The absorbable stent according to claim 1, wherein the teeth have a first surface with an angle of α1 to the outside surface and a second surface having and angle of α2 to the outside surface.

6. The absorbable stent according to claim 1,wherein α1 is greater than α2.

7. The absorbable stent according to claim 1, wherein the flexible covering has a plurality of openings to allow liquid to move between the central opening and the lumen of the blood vessel.

8. The absorbable stent according to claim 1, wherein the flexible covering has at least one opening to allow liquid to move between the central opening and a branch of the blood vessel.

9. The absorbable stent according to claim 8, wherein the flexible covering has a plurality of markers disposed around the at least one opening to allow for localization of the at least one opening relative to the branch of the blood vessel.

10. The absorbable stent according to claim 1, wherein the flexible covering is extruded from a flexible elastic co-polymer.

11. The absorbable stent according to claim 1, wherein the at least one expanding belt is comprised of Poly (lactide-co-glycolide), PLGA, in mole ratio of 50/50% to 95/5%, and most preferably 82/18%.

12. The absorbable stent according to claim 1, wherein the flexible covering and the at least one expanding belt is made of PLC in the molar ratio of 70:30%.

13. A method of inserting an absorbable stent in a blood vessel comprising:
providing the stent and a catheter having an expandable balloon, the stent having a flexible covering with a central opening extending between a first end and a second end and at least one expanding belt disposed within the central opening between the first end and the second end, the expanding belt having an inside surface and an outside surface and expanding radially in response to pressure exerted on the inside surface of the belt;
locating the stent on the expandable balloon on the catheter;
locating the stent and the expandable balloon at a lesion site in a blood vessel through a vessel opening;
expanding the expandable balloon, thereby expanding the stent at the lesion site;
deflating the expandable balloon; and
removing the expandable balloon from the blood vessel.

14. The method according to claim 13, further comprising the step of using a balloon catheter to compress the lesion site before locating the stent at the lesion site.

15. The method according to claim 13, further comprising the step of closing the vessel opening.

16. An absorbable stent to be inserted into a lumen of a blood vessel comprising:
a flexible covering having a central opening extending between a first end and a second end; and
a plurality of expanding belts disposed within and spaced along the central opening between the first end and the second end, each of the plurality of expanding belts having an inside surface and an outside surface and having a plurality of teeth disposed along the outside surface to engage a pawl disposed on each of the plurality of expanding belts and each of the plurality of expanding belts expanding radially in response to pressure exerted on the inside surface of the belt.
